(19) United States
(12) Patent Application Publication
(10) Pub. No.: US 2025/0375346 A1
(43) Pub. Date: Dec. 11, 2025

(54) MULTIPURPOSE THERMAL ACCESSORY FOR PELVIC PHYSIOTHERAPY

(71) Applicant: Tatiana DIP, SÃO PAULO (BR)

(72) Inventor: Tatiana DIP, SÃO PAULO (BR)

(21) Appl. No.: 18/874,092

(22) PCT Filed: Jun. 12, 2022
(86) PCT No.: PCT/BR2022/050216

§ 371 Date: Dec. 12, 2024

(51) Int. Cl. A61H 19/00

(52) U.S. Cl. CPC A61H 19/44; A61H 2201/0207; A61H 2201/0214; A61H 2201/0257; A61H 2201/0285; A61H 2201/1692

(57) ABSTRACT

The present order refers to a Pelvic Physiotherapy Multipurpose Thermal Accessory. The present invention combines typical functions of a vaginal dilator (when used vaginally) or butt plug with functions of a thermal bag (cold or hot). The effectiveness of the treatment is optimized, as the treatment is much faster compared to products without thermal effect. In addition, the use of this invention combined with heat and cold, as it is the shortest treatment time, encourages the patient to continue with the treatment, significantly avoiding treatment evasion (withdrawal). “MULTIPURPOSE THERMAL ACCESSORY OF PELVIC PHYSIOTHERAPY” (100), characterized by containing flexible body (01), tip (02), limiter (03) of the body (01), cap (04) of the body (01), cavity (05) inside the body (01), material to be inserted (15) in the cavity (05) with thermal properties.

### OBJECTIVES AND BASIS OF THE INVENTION

It is an object of the present invention to provide a thermal device (100) to aid in the treatment by reducing and eliminating pain via the vaginal or anal route.

In the post-operative period, the present invention with the use of cold helps to reduce swelling and treat edema and accelerate the patient's recovery, both vaginally and anally. The present invention is also of great importance in the preparation of the pregnant woman for childbirth and in the postpartum period, encouraging natural vaginal childbirth. During the postpartum period, vasoconstriction with cold also helps to accelerate the postpartum recovery of the pelvic region.

The present invention (100), when used in conjunction with heat, promotes vasodilation, improving the absorption of active ingredients in medicines (phytotherapeutics, anti-inflammatories, calming products and those that promote relaxation, among others) presented in creams and gels (faster absorption) or ointments (slower absorption, generally oily-based), optimizes muscle relaxation and accelerates local inflammation (both vaginally and anally).

Mainly via the anus, heat helps to release endorphins, and due to the increase in circulation, it brings more nutrients, more oxygen and more collagen to the region, which explains the acceleration in treatment with the use of this invention (100).

Other functions of this thermal accessory (100) that deserve to be highlighted include treating pain trigger points, especially in textured versions. Stretching and relaxing the pelvic floor muscles, when used as a dilator. When used as a dilator, its purpose is to educate, teach relaxation while promoting muscle stretching. Strengthening and harmonizing the pelvic floor muscles, through typical exercises and more modern and diverse pelvic physiotherapy exercises performed with this invention, using different levels of hardness.

Pelvic physiotherapy exercises also stimulate better vaginal lubrication, as scientific research has shown. The present object of this patent (100) also facilitates awareness and better perception, awareness of the local muscles, improving the patient's health and promoting a fuller sex life, having fuller, more complete sexual relations and with greater intensity of sexual pleasure.

The present invention (100), when used with intense heat, mainly allows vasodilation, relaxation, intensification of blood circulation, and local disinflammation to be highlighted.

When used in conjunction with cold, it mainly allows vasoconstriction, reduction of edema, and reduction of burning and stinging symptoms to be highlighted. Vasoconstriction is very useful in post-surgical cases, reducing swelling, treating edema, both in surgeries performed by proctologists and gynecologists, as well as in aesthetic treatments of the intimate region. Vasoconstriction is also very useful in women after childbirth, especially natural childbirth, aiming at a faster return to normality of all the pelvic muscles after childbirth, which were dilated during labor for the baby to be born. Postpartum exercises also rebalance and harmonize all the muscles.

Among the most diverse cases and uses, the present invention (100) is indicated for, via the vagina: vaginismus (impossibility of penetration or performing a gynecological examination due to involuntary contraction of the pelvic floor muscles); dyspareunia (pain during or after sexual intercourse); use during pregnancy and postpartum, oncological cases (cancer); improving physical comfort during sexual intercourse; aiding in the relaxation of the pelvic floor muscles by reducing muscle tension; promoting desensitization of the vaginal canal; overcoming the fear of vaginal penetration; history of sexual trauma: psychological problems and sexual health problems associated with vaginal penetration.

Among the most diverse cases and uses via the vagina, the present invention (100) is also indicated for vulvodynia or vestibulodynia: burning sensation in the vulvar region; post-gynecological surgery; shortened or narrow vaginal canal resulting from gynecological surgery, pre- and post-partum treatments together with pelvic physiotherapy during pregnancy; pelvic pain (endometriosis, oncological treatment, others); menstrual cramps; vaginal stenosis: reduction of vaginal elasticity, narrowing and adhesions resulting from pelvic radiotherapy or brachytherapy; recovering flexibility after pelvic floor surgery in healing and narrowing and/or shortening of the vagina; preventing adhesions and fibrosis; other conditions that require local muscle dilation; stimulating pelvic perception (improving awareness and sensitivity of the muscles to intensify pleasure during sexual intercourse), giving flexibility to vaginal tissue, improving flexibility; vulvar and vaginal atrophy in menopause; difficulty in breaking the hymen when it is of the compliant or fibrous type.

The following may also be cited among the cases and uses of this invention: endometriosis: syndrome characterized by the presence of the endometrium outside the uterine cavity, causing pain; various pelvic physiotherapy treatments; allowing the use of internal absorbents and application of vaginal medication; facilitating the performance of gynecological examinations such as the Pap smear and transvaginal ultrasound; agenesis and neovagina (construction and reconstruction of the vaginal canal); post-surgical sexual reassignment: sex change; narrowing of the vaginal/anal canal; other uses.

In cases via the anal route, the use of this invention (100) is recommended (supervision of use by a health professional is always recommended) for Anismus (pain in the anus), Proctitis (inflammation of the rectal mucosa), Pre and Post-Surgical Treatment of Hemorrhoids (it is worth highlighting here that in some cases of pre-surgical hemorrhoids with intense pain, the use of this invention is not recommended due to the severity of the case, always have a doctor or health professional analyze the case before its use by the patient), Treatment of Fissures with professional guidance, assist in the relaxation of the muscles of the pelvic floor and anal region by reducing local muscle tension, treatment of tendonitis of the local muscles of the colorectal region, promote desensitization of the anus, overcome the fear of anal penetration, post-operative anal and prostate surgery.

There are also cases via the anal route where the use of this invention is recommended: improving physical comfort during anal intercourse, anal dilator, narrow anal canal resulting from local surgery, anal stenosis: reduction of anal elasticity, narrowing and adhesions resulting from local radiotherapy or brachytherapy, recovering flexibility after anal surgery in healing and narrowing, preventing adhesions and fibrosis, other conditions that require local muscle dilation, stimulating anal perception (improving awareness and sensitivity of the anal muscles to avoid pain from excessive contraction of the anal muscles), giving flexibility to the anal tissue, various pelvic physiotherapy treatments, agenesis and neoanus, (construction and reconstruction of the anal canal), treatment of edema (purple), post-surgical narrowing of the anal canal, constipation in case of excessive contraction (tension) of the pelvic floor, in addition to other uses.

When used mainly textured, it promotes increased perception via the vagina. When used with textures (06) with rounded tips, it aims to treat pain trigger points. The tips of both the body (02) and the textures (06) are rounded to treat, and not pierce or hurt the patient. The entire design of the product is also careful to avoid sharp corners that can accumulate microorganisms, aiming to facilitate the cleaning of the product by the user.

It is preferentially recommended that this invention be used under the guidance or supervision of a healthcare professional, especially a pelvic physiotherapist (also known as a urogynecological physiotherapist). Several professionals, among them, can be cited and highlighted, who work together in a multidisciplinary group or separately, who can indicate and recommend the use of the product, in addition to pelvic physiotherapists, urogynecological physiotherapists, we can mention: psychologists, psychiatrists, sexologists, gynecologists, obstetricians, proctologists (when anal), nurses, caregivers, among others.

Furthermore, the present invention (100) can be used as a sex shop product to promote, stimulate and increase pleasure, in addition to recreational use. Intense thermal use, integrated or not with a vibrator, can also be used in several cases, mainly as a sex shop item.

### STATE OF ART

The current state of the art can be established through the following documents listed below:

Document U.S. Pat. No. 8,097,014 B2 relates to a variable stiffness vaginal dilation device comprising a catheter 8 having a plurality of ports 9a, an inflatable member or balloon 7, a tubular member 3, a valve and stopcock 5, a syringe 6 or pump 10, and optionally an in-line pressure gauge 11. At least a portion of the catheter 8 is disposed within the inflatable balloon 7. The entire catheter 8 is disposed within the inflatable balloon 7. One end of the catheter 8 extends within the balloon 7 and the other end of the catheter 8 is attached to the balloon 7 and to an end of the tubular member 3 in a fluid-tight (i.e., water and air) manner.

The other end of the tubular member 3 is attached to a valve or stopcock 5 in a fluid-tight (i.e., water and air) manner. There is an air- and liquid-tight seal between the tubular member 3 and the stopcock 5. The stopcock 5 communicates with a piston syringe 6 or with a pump 10 and an optional in-line pressure gauge 11. When the balloon 7 is inflated to the desired diameter and pressure, the stopcock 5 is closed so that the syringe can be removed and the inflated balloon 7 left in the vagina for the desired period of time.

Document CN108542342 A relates to a household vaginal dilator. The dilator comprises a sleeve (1) and a telescopic expansion mechanism installed in the sleeve (1), the telescopic expansion mechanism comprises a telescopic circular tube (2) and a sealing sliding ring (3), the telescopic circular tube (2) is inserted into the sealing sliding ring (3) and slides up and down in an inner sleeve (13), A plurality of expansion device assemblies (4) are arranged on the circumference of the working end of the telescopic circular tube (2), and an expansion device assemblies (4) includes an expansion rod (41) and a connecting rod (42), and further includes a transparent “air bag” (5).

The dilator ensures ease of operation in use by the cooperation of the expansion devices and the transparent “air bag”, the expansion of the working end device can be achieved by just pushing the telescopic circular tube, ease of operation at home is provided, in addition to the arrangement of a layer of lubricating fluid and a heating wire which further increases comfort, avoiding the cold and stiff sensations of traditional devices.

Document MU 8402394-5 refers to an anal dilator developed to facilitate the anal treatment of low rectoceles (at the level of the upper anal canal) using a mechanical stapler, characterized by the fact that it has a chamfer (1) in the tubular portion (4) of its structure. This dilator provides an increase in the area of the mucosa exposed, and allows the induction of this exposure through the anus, since the chamfer allows the manual reach of this technique. It has two ears (2) with two holes (3) each, which are used to fix the dilator in the anus, whose anatomical shape provides greater ease in the procedures, both for handling and for the fixation itself.

The dilator is inserted into the patient's anus with the aid of the introduction plunger (E), which has a triangular-shaped handle (5) and a cylindrical, pointed head (6), the diameter of which is designed so that the tube (4) of the dilator passes through it tightly, until it touches the handle.

A relates to anorectal dilators and medicators, and particularly to a device of this type which is adapted for use in connection with the treatment of disorders of the anorectal canal wherein it is desirable to dilate or expand the walls of the canal to a predetermined degree and simultaneously deliver medicament to the walls of the canal to treat affected areas thereof. A device is proposed which functions to simultaneously dilate the walls of the anorectal canal and deliver a treatment agent to the walls.

Such devices may be classified as syringes in which the fluid used to expand the sac is the same fluid used as the treatment agent, the fluid passing out of the sac through openings in it to contact the walls of the anorectal passage.

Document USD840029 S refers to a vaginal dilator without a seal made of rigid plastic, with a removable handle. This removable handle is an option for use by healthcare professionals. The object of this document is currently sold in e-commerce in Europe. The material of this product is rigid plastic. Many professionals do not recommend rigid plastic because if it breaks inside the body it can hurt the user, especially if the plastic is of the type that generates sharp or piercing splinters when broken.

Poor quality glass sex shop accessories are also banned for the same reason that they can break and become sharp or piercing inside the anus or vagina. In addition, the lack of sealing prevents the accumulation of internal liquid. It is made to use the handle only when inserting and removing.

There are other dilators on the market that are softer (compared to the hard plastic one mentioned), always one-piece (without an internal cavity), sold in pelvic physiotherapy stores, sex shops, and on the websites of some pelvic physiotherapists, generally imitating silicone, cheap plastics, rubber and also without the use of heat. Document EP1754503A1 (US2007043388A1) describes a one-piece dilator with markings showing how far it was inserted vaginally.

The market also offers kits with different sizes, diameters and heights of vaginal dilators according to the progress of overcoming vaginismus and dyspareunia. However, no vaginal dilator on the market has representative thermal characteristics that greatly optimize the treatment of vaginismus and dyspareunia.

There are metal anal plugs sold in sex shops and e-commerces specifically for this purpose to provide pleasure and a sensation of cold touch, however the cold from the sensitive heat of the metal (or good quality glass) is not enough to treat, only for excitement and pleasure through the sensation of touch.

It is also worth mentioning that the use of heat and cold on the outside of the body for relaxation and treatment of the musculoskeletal system is known in medical literature and orthopedic physiotherapy. Women also place a hot water bottle on the abdominal region in cases of colic to reduce pain. These hot water bottles are traditional, where hot water or ice is placed, and a lid (cap), or in newer models on the market, already made of sealed material with gel inside, and are easily found in pharmacies.

However, there is no thermal bag in the literature that helps introduce heat via the vagina or anus in order to promote thermal treatment, as this invention promotes, according to a study in all patent banks researched, as well as on internet and world trade websites, as well as medical literature.

We have confirmation that today, through websites of good doctors, the only resource available is to introduce heat into the body via the vagina or anus by injecting hot water through a syringe via the anus in treatments recommended by proctologists, in addition to the sitz bath, which has been known for many centuries.

Some erotic toys have a very soft electrical resistance, of low intensity, with plastic materials and inferior to silicone, but this heat does not have the function or the power necessary to treat the patient. Their only function is to improve and get closer to a more realistic male sexual organ, through optimization of thermal sensation, with sensible heat only to increase the temperature of the product and provide thermal comfort with an electric heating circuit, they do not have enough power to treat and deflate the body. They reach the maximum temperature of the human body. And if the power is increased, there is a great risk of accidents (both shock, short circuit, and serious burns via the vagina and anus) and further damage to the user's health.

The present invention does not cause any risk of accidents, testing the most comfortable temperature before vaginal or anal introduction, close to the thighs or abdomen, being much safer, as the thermal product only becomes increasingly close to body temperature after its vaginal or anal introduction, during the time it is used by the user, even providing much more intense heat that allows the effectiveness and optimization of the treatment.

Sex and civilizations and cultures also cite cases of vibrators throughout history. It is worth mentioning that according to sources on YouTube, Cleopatra used a type of vibrator with a sanded wooden tube and live bees. The Romans used an accessory with leather skin to make it more realistic and similar to the male sexual organ. The Greeks used a type of hard bread for pleasure. Sex in its fullness is part of human nature in several Western and Eastern civilizations, such as the well-known book of Kama Sutra.

There are many different types of vibrators in online sex shops, including the “Mini” and “Bullet”, “Personal”, “Rabbit”, “Butterfly”, “Magic wand”, “Clitoral sucker”, “G-spot vibrator”.

In relation to the entire state of the art and the global market for pelvic physiotherapy products and sex shop products, there is nothing to date that has integrated representative thermal properties (such as external thermal ice and gel bags that are external to the body) with those of a vaginal dilator or anal plug. The present invention combines the typical functions of a vaginal dilator (when used vaginally) or anal plug with the functions of a thermal bag (with cold or heat, depending on the application), with much better treatment efficacy. The present invention combines the function of the anal plug or traditional dilator with the thermal bag, respectively in the cases via the anal and vaginal routes. By combining the two together, the efficacy of the treatments is optimized, since the treatment time in the tests reaches one third of the time in relation to products without thermal effect via the vaginal route.

Furthermore, the use of the present invention combined with heat and cold, as it is the shortest treatment time, encourages the patient to continue with the treatment, significantly avoiding evasion (withdrawal) from the treatment. Unfortunately, the entire pelvic area, which is a part of the body that needs to be treated in relation to health, often because it is also an intrinsically erogenous region, and full of taboos, in addition to religious and cultural factors, often does not receive the necessary hygiene, asepsis, care and adequate health treatment care in the area. This product also aims to make life easier for people who have these difficulties, through its high treatment efficacy, such as in cases of dyspareunia and vaginismus.

Many cases of dyspareunia and vaginismus have their origin in repression in sexual education, where the patient grows up hearing bad things about sex, that sex is bad, that sex is dirty. On the other extreme, there are other unfortunately very common cases of victims of rape and other sexual crimes such as pedophilia, which often occur mainly within the family environment, as shown in the film “Lara's Silence”, also available on YouTube, often accompanied by violence and death threats in the event of seeking help and reporting the criminal, often a psychopath, which is very sad and often prevents the victim from seeking appropriate help.

The serious stories of human trafficking and forced prostitution with threats to kill family members, in addition to taking possession of the victim's identity documents, cause terrible harm to these victims, who, when they are lucky enough to be rescued, generally need a lot of psychological or psychiatric support to treat the after-effects of the spirit, in addition to treating the after-effects of the body.

To treat the physical pain of these victims of these worst crimes against a human being, this invention comes to help the victim at a stage where he/she accepts physical treatment, so that the victim can return as far as possible to a life closer to normal before the trafficking and sexual violence he/she suffered.

Sexual pain is also very present in women who are victims of war, who are raped as an instrument of war. Horrible psychological and physical pain that no one really likes to talk about, such is the psychological and physical horror.

These people are human beings, and they need appropriate psychological, psychiatric support and pelvic physiotherapy treatment.

Unfortunately, some cultures in the world still treat women as objects, worth less than camels in the eyes of society and even families, who sell their daughters in exchange for some animals (yes, we are in 2021 when we write this patent, for anyone who reads this patent at any time).

How could a woman have such pleasure? Without choosing her partner. Not to mention the brutal age difference and the total lack of passion and reciprocal love. Some tribal cultures still mutilate female genitalia so that the woman does not feel anything when being penetrated (what happens to the psychology of a mutilated woman?).

It is good that international entities act in favor of the rights of these women, and also children, in addition to men who are also victims of these cultures rooted in some societies, also fighting against all the crimes mentioned here.

If it is extremely difficult for even a very brave and financially independent woman in modern society to say that she suffered sexual violence, as shown in the cover story of Veja magazine in 2020, among so many other articles on the internet, in which the victim is blamed for her dress, for her wide and happy smile, and the criminal is exempt from punishment, it is even more difficult for a man to say, for example, that he suffered a crime of pedophilia in modern society, even in 2021.

It is more necessary than ever to air out ideas about sexual life and health in society. This invention also came within one of its main objectives to help victims of sexual violence and treat the pain resulting from this very serious crime that has existed since the most remote times.

It is also worth remembering that few men have the sensitivity and empathy necessary to marry, care for and love a woman who has been raped, who often suffers from dyspareunia or vaginismus, and is unable to provide her partner with a full sex life.

Treatment with the aid of the present invention, especially combined with intense heat, allows happiness much closer to at least the full sexual life desired by this couple after adequate treatment with a group of health professionals.

A woman who overcomes all this ends up having a much fuller and happier life, and psychologically she becomes stronger and better able to cope with her work, her personal life and her social life. The consequences of sexual violence on a victim affect her entire life in the broadest aspects and treating the pain is essential for the victim to recover her self-esteem and continue her life as close to normal as possible.

Dr Drauzio Varella, a doctor who is a national reference in Brazil, also mentions on his YouTube website that there are women who suffer from dyspareunia without any related reason.

The percentage of Brazilian women of sexually active age who suffer from dyspareunia in Brazil today is significant. Canadian studies and the MD Saúde website report that up to 20% of women experience pain during or after vaginal intercourse.

It is definitely necessary to treat such pain that almost no one has the courage to say it publicly. Almost no one goes around saying they are sexually impotent, neither men nor women. Sex with pain is horrible, it should not be continued before adequate treatment, so that the woman can have pleasure and get closer to her partner, instead of pain and distance, denial, also affecting the relationship in the psychological aspect, or self-abuse and much more pain in silence, crying in the dark, alone, when no one is around.

It is still necessary to spread the word to society that there is treatment for female sexual dysfunction, that there is pelvic physiotherapy, and much has been done in this regard with the help of the internet, conferences, and an increase in the number of pelvic physiotherapists in Brazil and around the world. In 2020, there are approximately 8,000 pelvic physiotherapists in Brazil, compared to nearly 3,000 10 years earlier, which demonstrates intense growth and a lot of research in the area. Many gynecologists are still unaware of pelvic physiotherapy.

The fact that the present invention is in its premium model in medical grade silicone, naturally hypoallergenic, allows disinfection in water with boiling water for an adequate time, in addition to normal asepsis with soap and water for daily use, inside the home, without the need for typical products found exclusively in clinics and hospitals.

Medical grade silicone is much higher in quality than the fake silicones found on the market, which cannot undergo high-level disinfection with boiling water in a domestic environment.

### DETAILED DESCRIPTION OF THE INVENTION

The device of the present invention will be described in detail with the aid of the attached figures.

The device (100) of the present invention consists mainly of a body (01), which has at one end a rounded tip (02) of the body (01), and at the other end a limiter (03) of the body (01), and a cavity (05) inside the body (01) to accommodate the thermal material (15) to be inserted into the cavity (05), and then this cavity is closed with a lid (cap), (04).

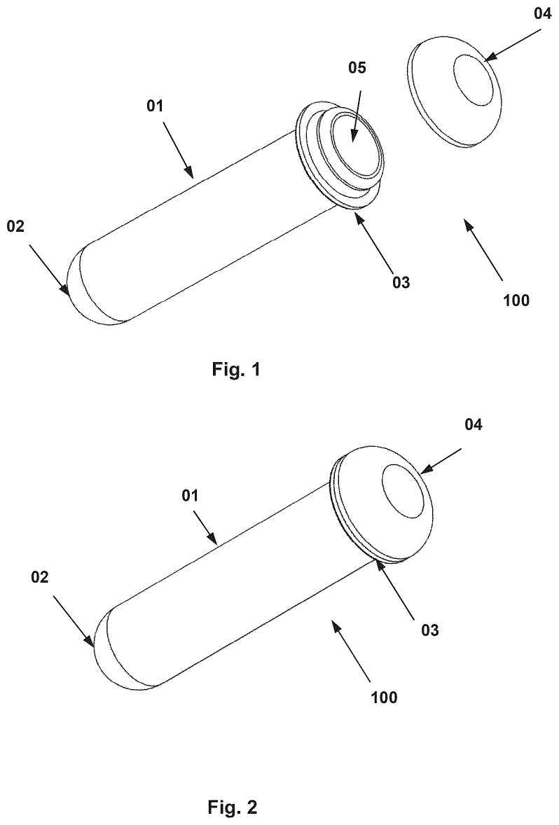

FIG. 1 shows an isometric view of the present invention (100) with preference for vaginal use with the lid (cap), (04) separated from the body (01).

FIG. 2 shows an isometric view of the present invention (100) with preference for vaginal use with the cap (04) already attached to the body (01).

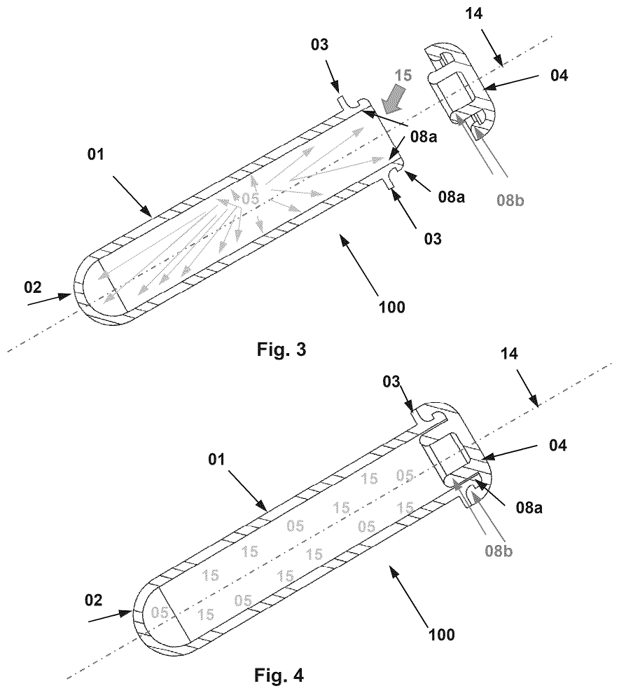

FIG. 3 shows a cross-sectional view of the present invention (100) with preference for vaginal use with the lid (cap) (04) separated from the body (01). In this FIG. 3 it is also possible to observe the central axis (14), the thermal material to be inserted (15) (NOT SHOWN), the body fitting (08a), with the lid (cap) fitting (08b), with the lid (cap, (04)) still separated from the body.

FIG. 4 shows a cross-sectional view of the present invention (100) with preference for vaginal use with the cap (04) already attached to the body (01). In this figure it is also possible to observe the central axis (14), the thermal material (NOT SHOWN) to be inserted (15), the body fitting (08a), with the cap fitting (08b). It is worth mentioning that medical grade silicone is the best quality material currently available on the market for the production of the body (01) and cap (04) and that to prevent the occurrence of leaks between the body (01) and the cap (04) the interlocking system shown was the best solution found since until the creation of this invention there was no silicone lock to seal liquids without threads, as far as we know. This was a technological challenge overcome with this invention.

Therefore, it is also worth describing this locking with cut o-rings as a new state of the art, but not limited to this locking system as the only one for the larger inventive concept. However, inferior copies of the main model described here are valid systems with a plastic thread in a second body complementary to the silicone body or the entire silicone body with thread, systems with the aid of a plastic preform and lid (cap), among others.

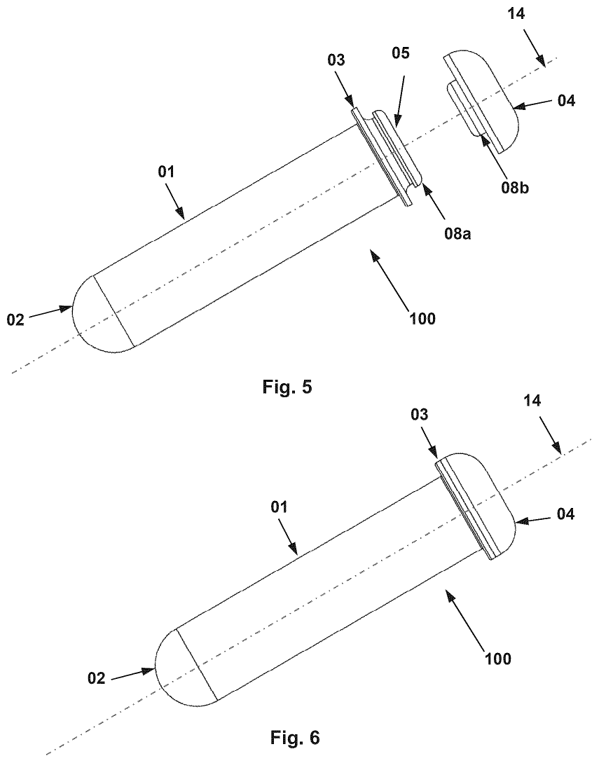

FIG. 5 shows a front view of the present invention (100) with preference for vaginal use with the lid (cap) (04) separated from the body (01).

FIG. 6 shows a front view of the present invention (100) with preference for vaginal use with the cap (04) already attached to the body (01).

It is worth remembering that in the cases of FIGS. 5 and 6 the products are symmetrical, the front and rear, top and bottom views are identical due to the axis of symmetry that passes through the center (01) of the body and the center of the lid (cap), (04).

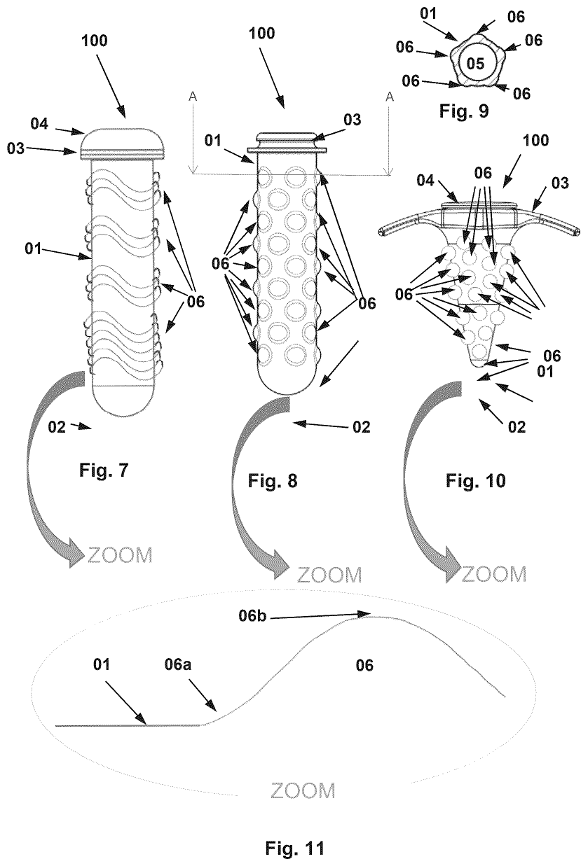

FIGS. 7, 8, 9, 10 and 11 illustrate the present invention (100) in three versions of possible textures (06) on the body (01), among the most diverse types of textures (06). FIG. 7 illustrates an example of texture (06) in curves on an accessory (100) for preferential vaginal use, with the zoomed-in profile in FIG. 10 showing in detail that this curve is all rounded and without sharp corners, aiming at the correct cleaning of the product, without sharp corners. The rounding is necessary for the patient's comfort and not to pierce or hurt the user's body.

FIG. 8 illustrates an example of a sphere texture (06) in an accessory (100) for preferential use via the vaginal route, also with the same zoom profile of the sphere texture (06) on the external wall of the body (01). FIG. 9 illustrates an example of a sphere texture (06) in an accessory (100) for preferential use via the anal route, also with the same zoom profile of the sphere texture (06) on the external wall of the body (01), FIG. 11. It is worth remembering that a measurement of close to 1 cm in sphere diameter is recommended via the vaginal route, but not limited to this measurement, and for the anal route, which is much more innervated (full of nerves), a measurement of close to 5 mm in sphere diameter is recommended, but also not limited to these measurements.

Texture (06) is important for removing pain trigger points, including when of the spherical type, in addition to improving perception (awareness of muscle contraction) and control and mastery over muscle contraction and relaxation.

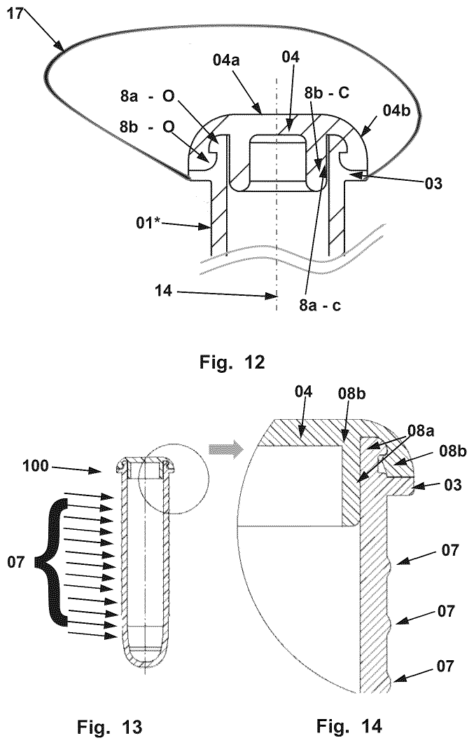

FIG. 12 shows in more detail and in section view the fit of the body (08 a) with the fit of the lid (cap) (08b). In this fit, it is worth noting that this fit profile is an innovation in the area of sealing in silicone parts with liquid storage. The O-ring cut into ¼ disc (08 a-O) in the body (01) is not at all obvious, with its mirrored opposite of ¼ O-ring (08 b-O) in the lid (cap) (04). And at the same time being only a sealing O-ring. Inside the lid (cap) (04), a lid (cap) cylinder (08 b-C) whose outer wall of the cylinder faces the inner wall of the body (08 a-C) also helps greatly to reduce leaks and optimize the sealing of the inserted material, for example hot water or ice and melted ice (cold water).

After this profile is developed, there may be other O-ring profiles, including with different numbers of O-rings and rectangular locks and in the most diverse formats, and this invention is not limited to this sealing system, but this sealing system is part of this technological advancement.

FIG. 12 also shows the lateral curve (04b) of the lid (cap) profile, when used mainly vaginally, being anatomical to the closing of the legs during use, without bothering the user, being comfortable, without pressing edges. It also shows an optional horizontal plateau (04a) on the lid (cap) (04) aiming at the display of the product in stands and fairs with the lid (cap) down and well supported on the table, in addition to also being anatomical for the user.

FIG. 12 also illustrates an example of a handle model (17) attached to the body (partially drawn, indicated as 1*), but is not limited to this. This handle is optional, easy to produce in the body mold and has the function of assisting in the removal of the accessory (100) from inside the vagina. Some pelvic physiotherapists prefer this handle in some types of patients. There are several types of handle (17) possible, permanent or removable, attached or not to the lid (cap) (04), and to the body (01). The lid (cap) (04) integrated with the handle (17) can have the most diverse shapes, including spherical, handle with spherical tip, handle similar to a sword dagger, among others.

The central axis (14) of this accessory (100) is verified and illustrated, and the body limiter (03) is also shown in greater detail, being intrinsic and inseparable from the body (01) in this example. The handle can also be part of the lid (cap) (04), or separate from the body (01) and the lid (cap) (04), that is, false and optional, as already exists on the market for conventional non-thermal expanders, without adding inventive matter, but adding to the state of the art.

FIG. 13, in addition to illustrating all the sealing details between the body (01) and cover (04) in a different section of two ¼ O-ring discs (the cover (04) has an internal “S” design different from the ¼ O-ring, but close), also illustrates depth markings (07) along the external surface of the body (01) in a section view of the entire accessory (100) and in zoom highlighted in the entire part by a disc selecting the highlighted area.

The seal between body (01) and lid (cap) (04) can be a double O-ring in two halves, in addition to infinite other examples besides the classic pair of threads in body and lid (cap). However, the ¼ O-ring has advantages in its industrial production compared to the thread, since the threaded part requires a more complex mold to remove the part, rotating the tool relative to the part within the manufacturing process of the part for the part (whether body (01) or lid (cap) (04), to detach from the tool, making industrial tools more expensive in relation to the state of the art sealing with ¼ O-ring achieved and described here).

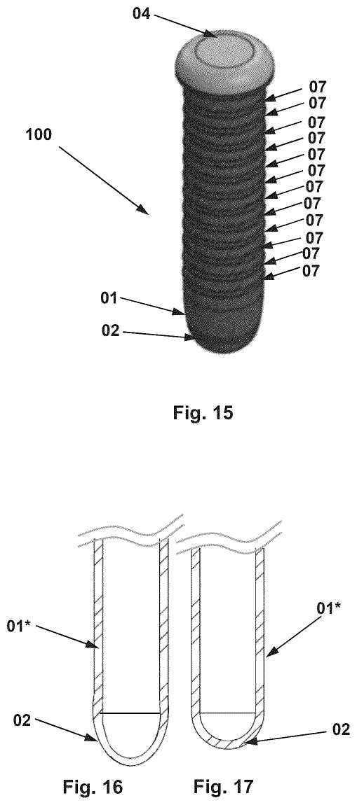

FIG. 15 describes the accessory (100) in isometric view with optional depth markings (07) along the body (01). The markings on the body here are on the surface adding material, and rounded like the textures for the same reasons of hygiene and not hurting the user. However, the depth markings can be made only with specific paint or even by removing or adding material from the body (01) and respecting good hygiene and asepsis practices, having curves and absence of sharp corners, valuing the premium quality of this product. The accessory (100) can optionally also be translucent or transparent, with or without colors, to facilitate the visualization and insertion of the thermal material to be inserted (15).

FIGS. 16 and 17 help to illustrate the tip (02) rounded to the body in its most typical forms. FIG. 16 illustrates a tip whose curve is an ellipse. This tip, especially for cases of vaginismus and dyspareunia, oncological cases and post-surgical narrowing of the vaginal canal, greatly aids the entry of the accessory (100) via the vagina. This type of elliptical tip is also highly recommended via the anus. FIG. 17 on the side shows a rounded tip that has the exact shape of a half circle. This type of tip is easier to manufacture, but is used more for cases of pregnancy, postpartum and the like. Both tip shapes (round and elliptical) are interesting and have advantages. The shapes of the tips can also be different from those mentioned.

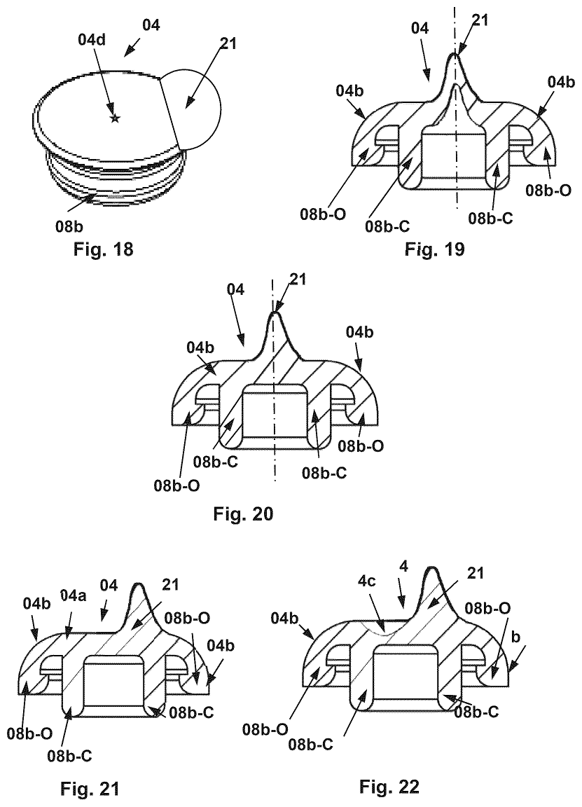

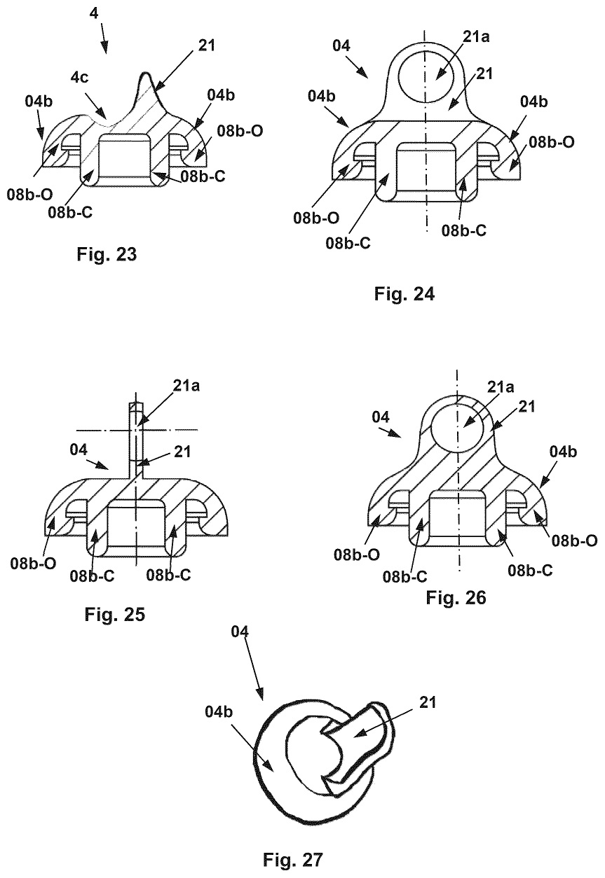

FIGS. 18, 19, 20, 21, 22, 23, 24, 25, 26 and 27 illustrate a lid (cap) (04) with a lid (cap) opening facilitator (21) and show in detail a double O-ring sealing system with the lid (cap) fitting (08b) being a double O-ring. This lid (cap) opening facilitator (21) (04) is important since the seal is strong and sometimes the user or healthcare professional has some difficulty in opening the lid (cap) without it, such is the quality of the closing of the system created by fitting the body (08 a), with fitting the lid (cap) (08 b) in the most diverse sealing systems mentioned here and possible, not only limited to those mentioned here in this document, but in the product leaflet the user can be instructed to press the body (01) close to the lid (cap) (04) and lift one side only by the end of the lid (cap) while the body is slightly pressed, facilitating the opening of the lid (cap).

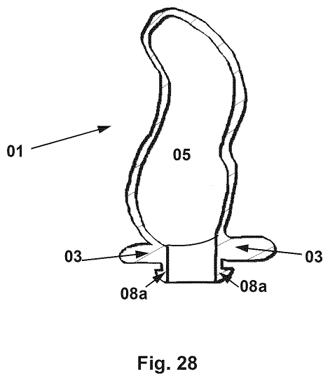

FIG. 28 illustrates some of the various typical vibrator shapes found on the market, which the body (01) of the accessory (100) can take, but is not limited to this. Note that the main axis (14) can be linear or curved, among other shapes, following the body. The body (01) can be conical, cylindrical, be overlapping spheres of various sizes, be a three-dimensional sword tip, among the most diverse shapes.

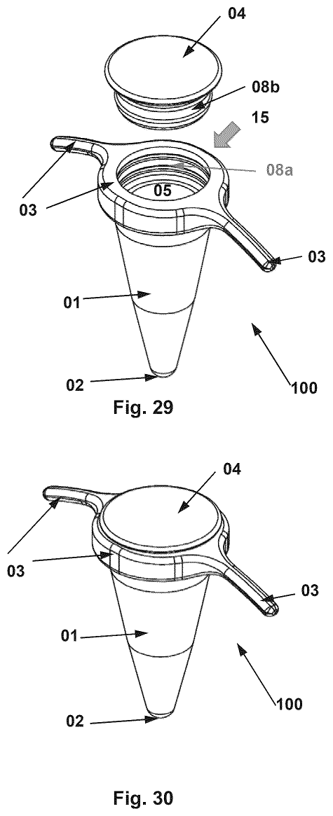

FIG. 29 shows an isometric view of the present invention (100) with preference for use via the anus with the cap (04) separated from the body (01).

FIG. 30 shows an isometric view of the present invention (100) with preference for use via the anus with the cap (04) already attached to the body (01).

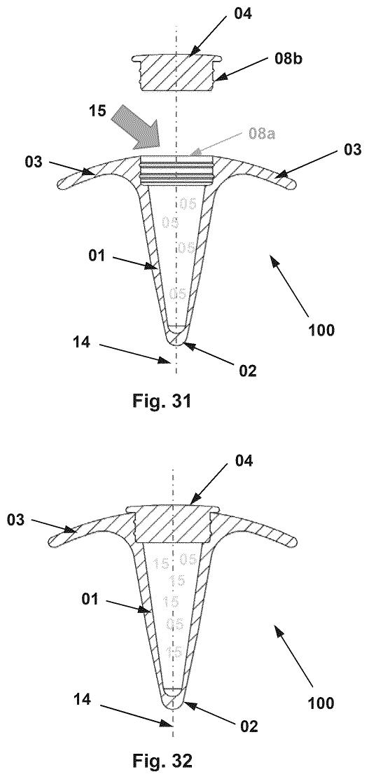

FIG. 31 shows a cross-sectional view of the present invention (100) with preference for anal use with the lid (cap) (04) separated from the body (01). In this figure it is also possible to observe the central axis (14), the thermal material to be inserted (15) (NOT SHOWN), the body fitting (08a), with the lid (cap) fitting (08b), with the lid (cap) still separated from the body.

FIG. 32 shows a cross-sectional view of the present invention (100) with preference for anal use with the lid (cap) (04) already attached to the body (01). In this figure it is also possible to observe the central axis (14), the thermal material (NOT SHOWN) to be inserted (15), the body fitting (08a), with the lid (cap) fitting (08b).

FIGS. 29, 31 and 32 show a double O-ring anal sealing system, which has another sealing system in relation to the sealing system illustrated in the accessory for preferential use via the vaginal route. In these FIGS. 29, 31 and 32, another possible sealing model is used for illustration, a double O-ring, however the sealing system is not restricted to this one, and may be different from this one and from the one already illustrated via the vaginal route. The system of a pair of threads in the cap and body is also possible, however it increases the cost of the tooling for the production of the molds, and is an inferior copy of the technology described above from the point of view of sealing between the body (01) and cap (04). It is also worth remembering that regardless of the model, the cap and the body can be sealed with or without the addition of material within the production process, and it is no longer possible to open them once sealed, as well as a variant of the process and product.

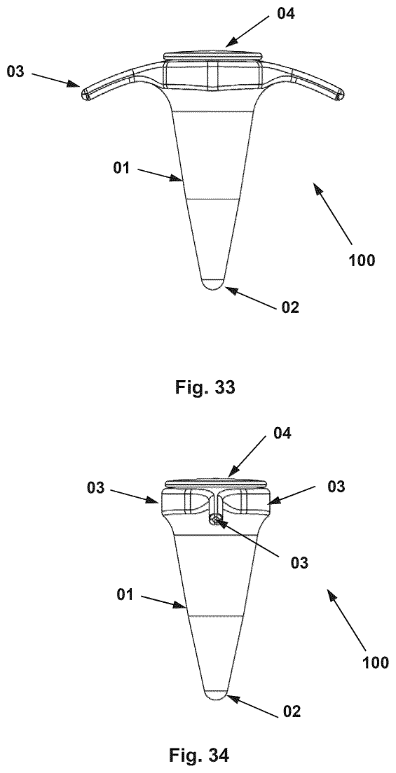

FIG. 33 shows a front view of the present invention (100) with preference for anal use with the cap (04) next to the body (01).

FIG. 34 shows a side view of the present invention (100) with preference for anal use with the cap (04) already attached to the body (01).

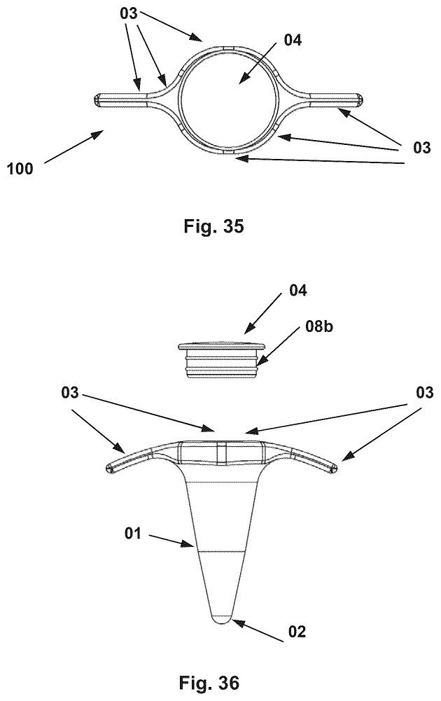

FIG. 35 shows a top view of the present invention (100) with preference for anal use with the cap (04) already attached to the body (01).

FIG. 36 shows a front view of the present invention (100) with preference for anal use with the cap (04) separated from the body (01).

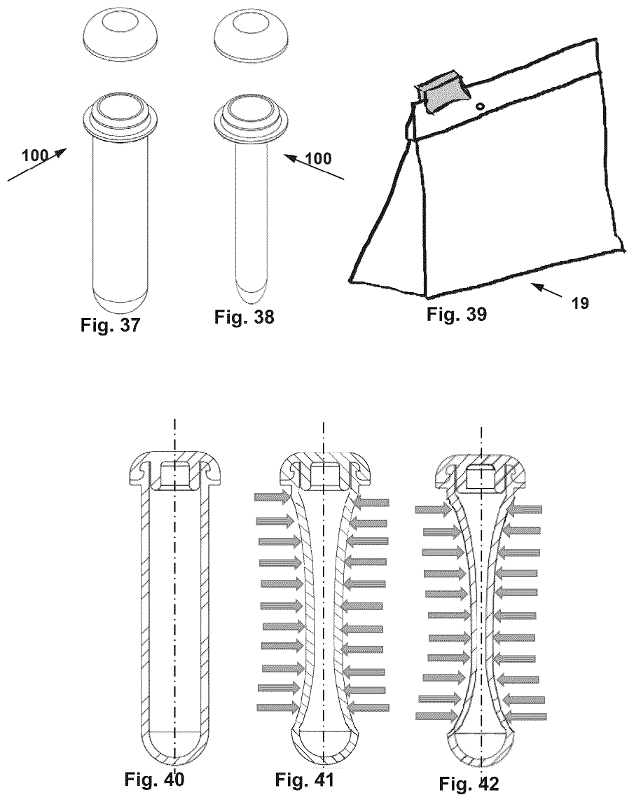

The set of FIGS. 37, 38 and 39 illustrate a kit (200) with two accessories (100) illustrated by FIG. 37 (larger diameter) and FIG. 38 (smaller diameter) and FIG. 39, which describes a toiletry bag (19), this preferably being entirely plastic or silicone, entirely washable and easy to dry with a towel, to store the kit (200).

The kits (200) can be very diverse, varying in different heights, different diameters, different hardness, different textures, different ways of use, different colors, having or not complementary rings, being only made of rings, among the most diverse combinations, facilitating the development of the patient's treatment. To save material, covers (22) can be made that are superimposed on the accessory. These covers are part of the accessory (100) and can be a hollow cylinder to be placed external to another existing body (01), or the body (01) with the tip (02) without the limiter (03) also to be placed external to another existing body (01), and have one or more different properties (the main ones being height, diameter, hardness, thickness, texture, color).

The set of FIGS. 40, 41 and 42 compares two accessories (100) of different hardness which, when tightened by the same force F (indicated by the gray arrows along the wall of the body (01)), deformation of the body occurs. different shape at different hardness. In other words, an accessory (100) of FIGS. 40 and 41 of all geometry identical to another accessory (100) illustrated in FIGS. 40 and 42, shown in FIG. 40 as being identical, deforms differently with the same applied force F.

The body in FIG. 41 has greater hardness than the body with the same geometry in FIG. 42 (body (01) in FIG. 42 has a lower shore hardness than the body (01) in FIG. 41). Therefore, body 41 presents greater resistance to the pressure of the distributed force, deforming the walls of the body (01) less compared to the body in FIG. 42, which deforms much more. This technical detail of changing the hardness of the product, which in silicone can, for example, be 50 shore A hardness and 80 Shore A hardness, is extremely important in the treatment of the patient, who can start the treatment with the lower hardness (for example 50 Shore A), and only then go to the 80 hardness accessory.

This is very important in completely atrophied and unbalanced muscles of the perineum, aiming to strengthen the local muscles via the vagina. It may be strange for a person who has never undergone such a treatment, but it is extremely important for the patient not to give up on treatment and to delay treatment. For those who have already been to the gym with dumbbells (weights) for the arms or shin pads in a weight training gym, the increasing hardness, in the case of the accessory, is equivalent to increasing the weights of the dumbbells or shin pads, only increasing the resistance to improve contraction and relaxation of the vaginal muscles.

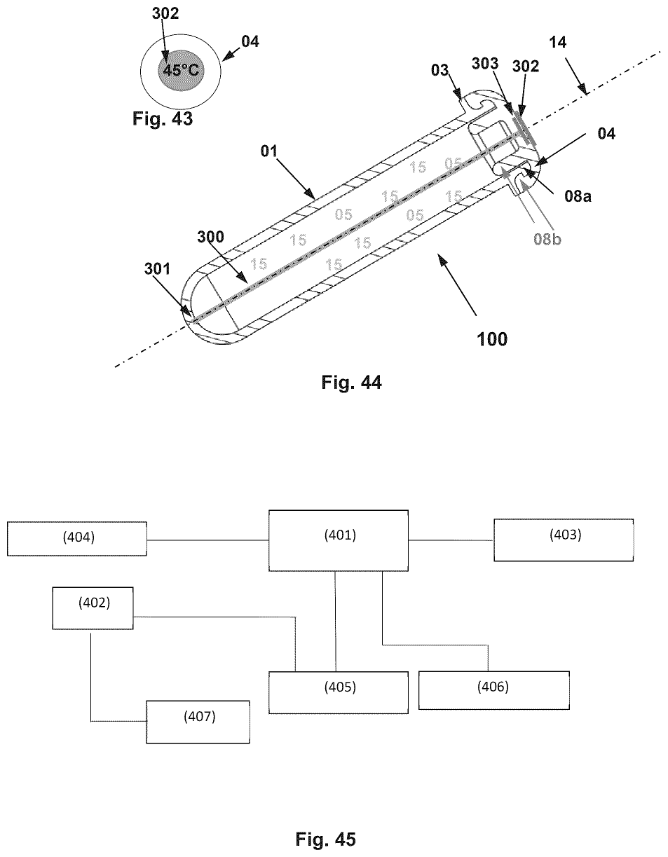

FIGS. 43 and 44 show a variant of the accessory (100) that has a thermometer (300) coupled to it. FIG. 43 shows a top view showing the thermometer display (302) and the accessory cover (04). FIG. 44 shows a sectional view of the accessory (100) with the thermometer (300), the thermometer display (302), a sealing adapter for the thermometer (303) and the tip (301) of the thermometer fixed to the tip (02) of the body (01). The thermometer can read the average temperature along the thermometer axis, or read a specific temperature point, depending on the model. The thermometer is also illustrated here with its axis going to the opposite end, however this is not necessary, as the thermometer can end internally at the lid (cap) (04) or go to any intermediate position between the lid (cap) (04) and the tip (02) of the accessory (100).

FIG. 45 describes one of several vibration systems (400) that can optionally be added to the accessory (100). In this example of a vibration system (400) we have a control board (401) with an internal microelectronic circuit, a battery (402) or other power source, a heating element (403) that can be, for example, an electrical resistance, or another element that generates heat or cooling, as well as, for example, a Peltier cell (typical of heating controller baths of sophisticated devices with the quality, for example, of laboratories that certify temperature control to give the INMETRO seal to equipment), or resistances and Peltier cells integrated and compatible with the system (400).

The heating system is optional and should be used with discretion when working above the human body temperature (36-37° C.) at the site, so as not to cause accidents, burns, fatalities or harm to the patient. There is no known heating system on the market above the human body temperature today, since the erotic toys on the market do not have a treatment function.

The electromechanical transducer (404) is responsible for generating vibration to the system and may be in the most diverse formats (plate, ring, others). It can generate the most diverse types of waves, pauses and vibration music. The adjustment buttons (405) aim to control the on and off, vibration intensity, vibration frequency, pulsation type and vibration type, heat or cold intensity, and may be directly on the cover (04) of the body (01) or on the remote control (406) and its respective optional physical device, wired or wireless, between the remote control and the body (01) of the accessory (100).

Since this entire vibration (400) and electric heating assembly is optional, and not the main part of the invention described here, we will not go into details about its most diverse variations, as the main part of this invention can be added to the most diverse types of vibrators available on the market.

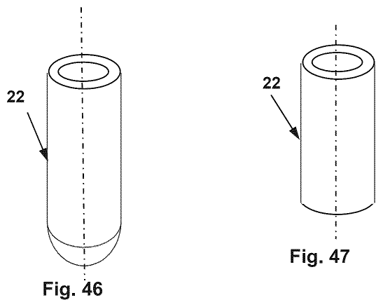

FIGS. 46 and 47 show two examples of a cover (22). FIG. 46 illustrates a cover (22) that covers the accessory (100), covering the body (01) and extending to the tip (02) of the body, covering the body (01) partially or completely, depending on the height of the body (01). FIG. 47 illustrates a cover that covers the body (01) partially, without covering the tip (02) of the body. Here the two covers (22) illustrated are smooth, but they can be textured, with depth markings, with different hardness, different colors, and all the other possible variant combinations.

The material to be inserted (15) into the cavity (05) so that it is fully inserted therein after the lid (cap) (04) is sealed with the body (01) can be inserted in the factory that produces the accessory (100), permanently sealing the lid (cap) (04) or having the option of being removed and replaced by the patient, with the lid (cap) (04) being removable.

The material to be inserted (15) can be liquid water at a temperature between 40° C. and 65° C., among other temperatures, it can be water with crushed ice, whole crushed ice in the shape of the cavity (05), a preferably three-dimensional sachet in the shape of the cavity (05) with a solution similar to thermal bags on the market (water+ cellulose acetate+optional pigments).

The material to be inserted (15) can also be a heating bag like those used to warm hands in cold countries (a supersaturated saline solution with a coin with a Gibbs energy burst (from thermodynamics variation of Gibbs energy (delta G)=variation of enthalpy (delta H)+T*(delta entropy)), this coin is an example of the starter (16), responsible for the beginning of crystallization and consequent emission of heat from the crystallization reaction, which can be endothermic (cools) or exothermic (heats)) without the risk of burning the patient, especially when the patient suffers from diabetes or other diseases that generate neuropathies.

The material to be inserted (15) may also be a wax or other solid material (a frozen solution) that melts in the patient or solidifies (in the case of wax and other examples) in the patient. In other words, the material to be inserted (15) may change phase during its use, in all its phase changes, mainly melting and solidification. The material to be inserted (15) may also be a gel.

The material to be inserted (15) can also undergo color change according to phase change and/or temperature, making it easier for the user to find the optimum point of use, with the body (01) preferably being transparent or translucent in this case.

The body (01) may optionally have reversible thermosensitive pigments, indicating the temperature variation for the patient. Reversible thermosensitive pigments are known from beer labels, to indicate the optimum temperature, on the ice cream scoop of a well-known brand in Brazil in a promotional campaign years ago (red or white), among several other uses.

The preferred material of the accessory (100) is medical grade silicone, which allows high-level disinfection in home use, in addition to cleaning with soap and water, and is a naturally hypoallergenic product. And this material can easily have its hardness changed with the addition of filler and pigments. The use of additives to pure silicone can optimize other desired properties of the material according to use. It is an inferior copy, but technically and economically possible, of the “fake silicone” plastic material that imitates silicone, also called “silicone” in the market.

Other possible and inferior materials are natural rubber (as it is porous), rigid and flexible plastics, metal and glass, thermoplastics and thermosets in general. The material of the accessory (100) can be manufactured in a blend with one or more of the materials previously mentioned.

A blend that deserves to be highlighted is medical grade silicone with added metal to improve thermal conductivity characteristics and improve and intensify the heat flow between the material to be inserted and the patient's body.

The accessory (100) was made for humans, but can be easily adapted for veterinary use, including post-surgery and oncological use in small animals (examples: dogs and cats), large animals (examples: horses, cows), wild animals (examples: lion, tiger, panda).

The accessory (100) is generally inserted by the patient herself. The handle (17) is already optional when a health professional removes the accessory from the patient's body. Another option, the handle (17) is characterized by clamps (18) to facilitate the removal of the accessory (100) from the patient's body, some examples of which are clamps (18) with claws (19), such as steakhouse clamps, clamps (18) that fit into the body (01), such as those available on the market for traditional one-piece dilators, clamps (18) that fit into the cap (04).

The body limiter (03) for vaginal use preferably has, among several possible shapes, a ring shape. For anal use, it preferably has the shape of an “I” intersected by a circle in its center, so as to fit in the cleft of the buttocks, between the buttocks, rounded, to cause the patient minimal discomfort, being as anatomical and comfortable as possible.

The lid (cap) (04) may optionally have an air vent to improve sealing of the assembly. This air vent must be a micro-hole to allow air to pass through but not the material to be inserted. The lid (cap) (04) may also be decorated with ornaments (plush toys, decorations) when used as a sexual fetish. The accessory (100) may also have a suction cup (24), preferably attached to the body (01) or attached to the lid (cap) (04).

To avoid wounds and injuries, it is recommended that the accessory be used with the aid of a lubricant (20). When made with premium quality, the accessory (100) receives treatment on the surface of the mold where it is produced, resulting in anti-static polishing on its surface. The lubricant (20) can be formulated together with a product with allopathic, homeopathic, phytotherapeutic, hormonal, anesthetic functions, among others, with the function of treating the patient.

There are several medications that are administered via the anus due to their rapid absorption, especially when the stomach is already very damaged. It is also recommended that the accessory be used protected by a condom, especially when administered anally, in order to avoid contamination of the accessory (100) and its complements with microorganisms and odors from the application site.

It is important to remember that the vagina has its own protective flora and is “clean” when healthy, while the anal tract is “dirty” and full of microorganisms and feces, depending on the diet and intestinal flora. In other words, if the vagina is healthy, it will not leave an odor on the accessory after it is washed with soap and water and with little effort. However, the anal tract without a condom can leave odors and residues on the product more easily, for the same quality of washing. Without a condom, after anal use, careful washing is recommended to avoid residues and odors on the accessory (100). In other words, the condom makes hygiene of the product much easier.

The accessory (100) can be made of material with antimicrobial properties, aiming for greater durability between two washes with water and soap, properly dried and stored, as well as properties that optionally protect against ultraviolet rays, if exposed to sunlight.

The accessory can be characterized by the tip (02) designed to have the shape of a spherical arch, making it easier to manufacture. The shape of the tip (02) in the shape of an elliptical arch has the function of facilitating the anal or vaginal introduction into the patient's body.

The accessory (100) can, during its industrialization, produce the lid (cap) (04) with a different hardness than the body (01), optionally, aiming at a better sealing of the material (15) to be inserted inside the cavity. This difference in hardness is typical of the beverage industry, mainly soft drinks, where the sealing material, integrated or not into the lid (cap) of the soft drink, has a different hardness than the nozzle of the PET bottle.

1. “MULTI-PURPOSE THERMAL ACCESSORY FOR PELVIC PHYSIOTHERAPY” (100), characterized by having a flexible body (01), tip (02) rounded to the body (01), limiter (03) of the body (01), lid (04) of the body (01), cavity (05) inside the body (01), material to be inserted (15) in the cavity (05) with thermal properties.

2. “MULTI-PURPOSE PELVIC PHYSIOTHERAPY THERMAL ACCESSORY” (100), according to claim 1, characterized by having the body (08 A) fit with the lid (08 B) fitting, aiming at the sealing between both so that the material to be inserted (15) is completely kept in the cavity (05).

3. “MULTI-PURPOSE PELVIC PHYSIOTHERAPY THERMAL ACCESSORY” (100), according to claims 1 and 2, characterized by the body (01) having textures (06).

4. “MULTI-PURPOSE PELVIC PHYSIOTHERAPY THERMAL ACCESSORY” (100), according to claims 1, 2 and 3, characterized by the body (01) having textures (06), being an example textures (06) in the shape of rounded discs.

5. “MULTI-PURPOSE PELVIC PHYSIOTHERAPY THERMAL ACCESSORY” (100), according to claims 1, 2 and 3, characterized in that the body (01) has textures (06), for example textures (06) in the shape of parallel waves.

6. “MULTI-PURPOSE PELVIC PHYSIOTHERAPY THERMAL ACCESSORY” (100), according to claims 1 and 2, characterized in that the body (01) has depth markings (07) along its central axis (14).

7. “MULTI-PURPOSE PELVIC PHYSIOTHERAPY THERMAL ACCESSORY” (100), according to claims 1, 2, 3, 4, 5 and 6, characterized in that the material to be inserted (15) is liquid.

8. “MULTI-PURPOSE PELVIC PHYSIOTHERAPY THERMAL ACCESSORY” (100), according to claims 1, 2, 3, 4, 5, 6 and 7, characterized in that the liquid material to be inserted (15) is drinking water at its most diverse temperatures.

9. “MULTI-PURPOSE PELVIC PHYSIOTHERAPY THERMAL ACCESSORY” (100), according to claims 1, 2, 3, 4, 5 and 6, characterized in that the material to be inserted (15) is gel.

10. “MULTI-PURPOSE PELVIC PHYSIOTHERAPY THERMAL ACCESSORY” (100), according to claims 1, 2, 3, 4, 5, 6 and 9, characterized in that the material to be inserted (15) in gel state is a solution of water with cellulose acetate.

11. “MULTI-PURPOSE PELVIC PHYSIOTHERAPY THERMAL ACCESSORY” (100), according to claims 1, 2, 3, 4, 5, 6, 7, 8, 9 and 10, characterized in that the material to be inserted (15) is a water solution with non-toxic salts.

12. “MULTI-PURPOSE PELVIC PHYSIOTHERAPY THERMAL ACCESSORY” (100), according to claims 1, 2, 3, 4, 5 and 6, characterized in that the material to be inserted (15) is solid.

13. “MULTI-PURPOSE THERMAL ACCESSORY FOR PELVIC PHYSIOTHERAPY” (100), according to claims 1, 2, 3, 4, 5, 6 and 12, characterized in that the solid material to be inserted (15) is, for example, drinking water ice in its most diverse forms, among them being whole, shaved, in stones.

14. MULTI-PURPOSE THERMAL ACCESSORY FOR PELVIC PHYSIOTHERAPY” (100), according to claims 1, 2, 3, 4, 5, 6, 10, 12 and 13, characterized in that the solid material to be inserted (15) is water ice with non-toxic salts.

15. “MULTI-PURPOSE THERMAL ACCESSORY FOR PELVIC PHYSIOTHERAPY” (100), according to claims 1, 2, 3, 4, 5, 6, 10, 12 and 13, characterized in that the solid material to be inserted (15) is water ice with cellulose acetate.

16. “MULTI-PURPOSE THERMAL ACCESSORY FOR PELVIC PHYSIOTHERAPY” (100), according to claims 1 to 15, characterized in that the material to be inserted (15) undergoes a phase change during its use.

17. “MULTI-PURPOSE THERMAL ACCESSORY FOR PELVIC PHYSIOTHERAPY” (100), according to claims 1 to 16, characterized in that the material to be inserted (15) is packaged in a sachet (16).

18. “MULTI-PURPOSE THERMAL ACCESSORY FOR PELVIC PHYSIOTHERAPY” (100), according to claims 1 to 17, characterized in that the material to be inserted (15) changes color according to the temperature.

19. “MULTI-PURPOSE THERMAL ACCESSORY FOR PELVIC PHYSIOTHERAPY” (100), according to claims 1 to 18, characterized in that the sachet (16) is three-dimensional, following the cavity (05).

20. “MULTI-PURPOSE THERMAL ACCESSORY FOR PELVIC PHYSIOTHERAPY” (100), according to claims 1 to 19, characterized in that the material (15) to be inserted into the cavity is a supersaturated solution of salts that are crystallized with a starter element (16).

21. “MULTI-PURPOSE PELVIC PHYSIOTHERAPY THERMAL ACCESSORY” (100), according to claims 1 to 20, characterized in that it is made of silicone material.

22. “MULTI-PURPOSE PELVIC PHYSIOTHERAPY THERMAL ACCESSORY” (100), according to claims 1 to 21, characterized in that it is made of medical grade silicone material.

23. “MULTI-PURPOSE PELVIC PHYSIOTHERAPY THERMAL ACCESSORY” (100), according to claims 1 to 22, characterized by being made of silicone material with filler additives in its formulation.

24. “MULTI-PURPOSE PELVIC PHYSIOTHERAPY THERMAL ACCESSORY” (100), according to claims 1 to 23, characterized by being made of silicone material and its blends.

25. “MULTI-PURPOSE PELVIC PHYSIOTHERAPY THERMAL ACCESSORY” (100), according to claims 1 to 20, characterized by being made of thermosetting material and its blends.

26. “MULTI-PURPOSE PELVIC PHYSIOTHERAPY THERMAL ACCESSORY” (100), according to claims 1 to 20, characterized by being made of thermoplastic material and its blends.

27. “MULTI-PURPOSE PELVIC PHYSIOTHERAPY THERMAL ACCESSORY” (100), according to claims 1 to 20, characterized by being made of elastomer material.

28. “MULTI-PURPOSE PELVIC PHYSIOTHERAPY THERMAL ACCESSORY” (100), according to claims 1 to 20, characterized by being made of suitable glass.

29. “MULTI-PURPOSE PELVIC PHYSIOTHERAPY THERMAL ACCESSORY” (100), according to claims 1 to 20, characterized by being made of metal.

30. “MULTI-PURPOSE THERMAL ACCESSORY FOR PELVIC PHYSIOTHERAPY” (100), according to claims 1 to 29, characterized by being made in a composition whose formulation is a partial to total mixture of all possible materials mentioned in claims 20 to 29.

31. “MULTI-PURPOSE THERMAL ACCESSORY FOR PELVIC PHYSIOTHERAPY” (100), according to claims 1 to 30, characterized by having a vaginal dilator function.

32. “MULTI-PURPOSE THERMAL ACCESSORY FOR PELVIC PHYSIOTHERAPY” (100), according to claims 1 to 30, characterized by having a vaginal vasoconstrictor function.

33. “MULTI-PURPOSE PELVIC PHYSIOTHERAPY THERMAL ACCESSORY” (100), according to claims 1 to 30, characterized by having an anal dilator function.

34. “MULTI-PURPOSE PELVIC PHYSIOTHERAPY THERMAL ACCESSORY” (100), according to claims 1 to 30, characterized by having an anal vasoconstrictor function.

35. “MULTI-PURPOSE THERMAL ACCESSORY FOR PELVIC PHYSIOTHERAPY” (100), according to claims 1 to 30, characterized by having an anal plug function.

36. “MULTI-PURPOSE THERMAL ACCESSORY FOR PELVIC PHYSIOTHERAPY” (100), according to claims 1 to 35, characterized by being used in humans.

37. “MULTI-PURPOSE THERMAL ACCESSORY FOR PELVIC PHYSIOTHERAPY” (100), according to claims 1 to 35, characterized by being used in animals.

38. “MULTI-PURPOSE PELVIC PHYSIOTHERAPY THERMAL ACCESSORY” (100), according to claims 1 to 38, characterized by being used at an adequate temperature, with a heat source coming from the material to be inserted (15) into the cavity (05).

39. “MULTI-PURPOSE PELVIC PHYSIOTHERAPY THERMAL ACCESSORY” (100), according to claims 1 to 39, characterized by changing color according to the temperature.

40. “MULTI-PURPOSE PELVIC PHYSIOTHERAPY THERMAL ACCESSORY” (100), according to claims 1 to 40, characterized by changing color according to the temperature, with reversible heat-sensitive pigments.

41. “MULTI-PURPOSE PELVIC PHYSIOTHERAPY THERMAL ACCESSORY” (100), according to claims 1 to 41, characterized in that the body (01) has different hardnesses to optimize health treatment.

42. “MULTI-PURPOSE PELVIC PHYSIOTHERAPY THERMAL ACCESSORY” (100), according to claims 1 to 42, characterized in that the central axis (14) of the body (01) is linear.

43. “MULTI-PURPOSE PELVIC PHYSIOTHERAPY THERMAL ACCESSORY” (100), according to claims 1 to 42, characterized in that the central axis (14) of the body (01) is curved.

44. “MULTI-PURPOSE PELVIC PHYSIOTHERAPY THERMAL ACCESSORY” (100), according to claims 1 to 44, characterized in that the body (01) is cylindrical.

45. “MULTI-PURPOSE PELVIC PHYSIOTHERAPY THERMAL ACCESSORY” (100), according to claims 1 to 44, characterized in that the body (01) is conical.

46. “MULTI-PURPOSE PELVIC PHYSIOTHERAPY THERMAL ACCESSORY” (100), according to claims 1 to 44, characterized in that the body has the most diverse shapes.

47. “MULTI-PURPOSE PELVIC PHYSIOTHERAPY THERMAL ACCESSORY” (100), according to claims 1 to 47, characterized by the body (01) having the most diverse shapes, for example interconnected spheres.

48. “MULTI-PURPOSE PELVIC PHYSIOTHERAPY THERMAL ACCESSORY” (100), according to claims 1 to 48, characterized by the fitting of the body (08A) together with the fitting of the lid (08B) having the most diverse forms of sealing, for example, but not limited to, thread and counter thread (also called male thread and female thread or neck thread and lid thread), mechanical lock formed by an o-ring and its reflection (negative) for fitting in its most diverse variations in quantity of o-rings, and cutting of o-rings (half, quarter and the like), for example, those assembled under pressure.

49. “MULTI-PURPOSE PELVIC PHYSIOTHERAPY THERMAL ACCESSORY” (100), according to claims 1 to 49, characterized in that the body (08 A) fits together with the lid (08 B) and is sealed after the material to be inserted (15) into the cavity (05) during the manufacturing process.

50. “MULTI-PURPOSE PELVIC PHYSIOTHERAPY THERMAL ACCESSORY” (100), according to claims 1 to 50, characterized in that the handle (17) is fixed to the body (01).

51. “MULTI-PURPOSE THERMAL ACCESSORY FOR PELVIC PHYSIOTHERAPY” (100), according to claims 1 to 51, characterized in that the handle (17) is fixed to the lid (04).

52. “MULTI-PURPOSE THERMAL ACCESSORY FOR PELVIC PHYSIOTHERAPY” (100), according to claims 1 to 51, characterized in that the clamp (18) facilitates the removal of the accessory (100) from the patient's body, some examples of which are clamps (18) with claws (19), clamps (18) that fit into the body (01), clamps (18) that fit into the lid (04).

53. “MULTI-PURPOSE PELVIC PHYSIOTHERAPY THERMAL ACCESSORY” (100), according to claims 1 to 52, characterized in that the limiter (03) of the body (01) has a ring shape.

54. “MULTI-PURPOSE PELVIC PHYSIOTHERAPY THERMAL ACCESSORY” (100), according to claims 1 to 53, characterized in that the limiter (03) of the body (01) has an “I” shape, resembling a “T” together with the body (01).

55. “MULTI-PURPOSE PELVIC PHYSIOTHERAPY THERMAL ACCESSORY” (100), according to claims 1 to 54, characterized in that the limiter (03) is anatomical.

56. “MULTI-PURPOSE THERMAL ACCESSORY FOR PELVIC PHYSIOTHERAPY” (100), according to claims 1 to 55, characterized in that the limiter (03) has rounded corners for greater patient comfort.

57. “MULTI-PURPOSE THERMAL ACCESSORY FOR PELVIC PHYSIOTHERAPY” (100), according to claims 1 to 56, characterized in that the accessory (100) is arranged in kits (200), among its most diverse examples, with more than one unit of the accessory (100).

58. “MULTI-PURPOSE THERMAL ACCESSORY FOR PELVIC PHYSIOTHERAPY” (100), according to claims 1 to 57, characterized in that the Kit (200), among its most diverse examples, has two or more accessories (100) of different hardness.

59. “MULTI-PURPOSE PELVIC PHYSIOTHERAPY THERMAL ACCESSORY” (100), according to claims 1 to 58, characterized by the Kit (200), among its most diverse examples, having two or more accessories (100) of different textures.

60. “MULTI-PURPOSE PELVIC PHYSIOTHERAPY THERMAL ACCESSORY” (100), according to claims 1 to 59, characterized by the Kit (200), among its most diverse examples, having two or more accessories (100) of different sizes, examples being different diameters, different heights, cross-section of different dimensions.

61. “MULTI-PURPOSE PELVIC PHYSIOTHERAPY THERMAL ACCESSORY” (100), according to claims 1 to 60, characterized in that the Kit (200), among its most diverse examples, has two or more accessories (100) of different formats.

62. “MULTI-PURPOSE PELVIC PHYSIOTHERAPY THERMAL ACCESSORY” (100), according to claims 1 to 61, characterized in that the Kit (200), among its most diverse examples, has two or more accessories (100) of different functions.

63. “MULTI-PURPOSE PELVIC PHYSIOTHERAPY THERMAL ACCESSORY” (100), according to claims 1 to 62, characterized in that the lid has an air vent (23).

64. “MULTI-PURPOSE THERMAL ACCESSORY FOR PELVIC PHYSIOTHERAPY” (100), according to claims 1 to 63, characterized in that the accessory (100) or kit (200) is accompanied by a toiletry bag (19).

65. “MULTI-PURPOSE THERMAL ACCESSORY FOR PELVIC PHYSIOTHERAPY” (100), according to claims 1 to 64, characterized in that the toiletry bag (19) is plastic.

66. “MULTI-PURPOSE THERMAL ACCESSORY FOR PELVIC PHYSIOTHERAPY” (100), according to claims 1 to 65, characterized in that the lid (04) is anatomical to the patient's body.

67. “MULTI-PURPOSE PELVIC PHYSIOTHERAPY THERMAL ACCESSORY” (100), according to claims 1 to 66, characterized in that the lid (04) is anatomical to the patient's body.

68. “MULTI-PURPOSE PELVIC PHYSIOTHERAPY THERMAL ACCESSORY” (100), according to claims 1 to 67, characterized in that the lid (04) is curved on the sides and straight at the upper end.

69. “MULTI-PURPOSE PELVIC PHYSIOTHERAPY THERMAL ACCESSORY” (100), according to claims 1 to 68, characterized in that the accessory (100) is used with the aid of a lubricant (20).

70. “MULTI-PURPOSE THERMAL ACCESSORY FOR PELVIC PHYSIOTHERAPY” (100), according to claims 1 to 69, characterized in that the accessory (100) has an anti-static polished surface.

71. “MULTI-PURPOSE THERMAL ACCESSORY FOR PELVIC PHYSIOTHERAPY” (100), according to claims 1 to 70, characterized in that the accessory (100) has a suction cup (24), preferably attached to the body (01) or the lid (04).

72. “MULTI-PURPOSE THERMAL ACCESSORY FOR PELVIC PHYSIOTHERAPY” (100), according to claims 1 to 71, characterized in that the accessory (100) receives additives in its material formulation, in order to have antimicrobial properties.

73. “MULTI-PURPOSE THERMAL ACCESSORY FOR PELVIC PHYSIOTHERAPY” (100), according to claims 1 to 72, characterized in that the accessory (100) receives additives in its material formulation, so as to have properties that protect against ultraviolet rays.

74. “MULTI-PURPOSE THERMAL ACCESSORY FOR PELVIC PHYSIOTHERAPY” (100), according to claims 1 to 73, characterized by the tip (02) projected in view having the shape of a spherical arc.

75. “MULTI-PURPOSE THERMAL ACCESSORY FOR PELVIC PHYSIOTHERAPY” (100), according to claims 1 to 74, characterized by the tip (02) projected in view having the shape of an elliptical arc, with the function of facilitating the anal or vaginal introduction into the patient's body.

76. “MULTI-PURPOSE THERMAL ACCESSORY FOR PELVIC PHYSIOTHERAPY” (100), according to claims 1 to 75, characterized by the lid (04) having an opening facilitator (21) when the user (patient or health professional) wishes to open the lid (04) to access the material inserted (15) inside the cavity (05).

77. “MULTI-PURPOSE THERMAL ACCESSORY FOR PELVIC PHYSIOTHERAPY” (100), according to claims 1 to 76, characterized by having a complementary cover (22) that fits onto the body (01).

78. “MULTI-PURPOSE THERMAL ACCESSORY FOR PELVIC PHYSIOTHERAPY” (100), according to claims 1 to 77, characterized in that the cover (22) has at least one of the properties (hardness, texture, size, shape) different from the body (01).

79. “MULTI-PURPOSE THERMAL ACCESSORY FOR PELVIC PHYSIOTHERAPY” (100), according to claims 1 to 78, characterized in that the cover (04) has a different hardness from the body (01), optional, aiming at a better sealing of the material (15) to be inserted inside the cavity.

80. “MULTI-PURPOSE PELVIC PHYSIOTHERAPY THERMAL ACCESSORY” (100), according to claims 1 to 79, characterized in that the Kit (200), among its most diverse examples, has at least one cover (22) and one accessory (100).

81. “MULTI-PURPOSE PELVIC PHYSIOTHERAPY THERMAL ACCESSORY” (100), according to claims 1 to 80, characterized in that the Kit (200), among its most diverse examples, has at least two covers (22).

82. “MULTI-PURPOSE PELVIC PHYSIOTHERAPY THERMAL ACCESSORY” (100), according to claims 1 to 81, characterized in that the Kit (200) has, among its most diverse examples, at least one gel bag as the material to be inserted.

83. “MULTI-PURPOSE THERMAL ACCESSORY FOR PELVIC PHYSIOTHERAPY” (100), according to claims 1 to 82, characterized by the lid (04) having a material relief (24).

84. “MULTI-PURPOSE THERMAL ACCESSORY FOR PELVIC PHYSIOTHERAPY” (100), according to claims 1 to 83, characterized by adding or mixing testosterone to the lubricant or already being the accessory's own lubricating cream or gel when used in some cases via the vaginal route, aiming to improve the effectiveness of the accessory product in the treatment, mainly in cases of dyspareunia and vaginismus.

85. “MULTI-PURPOSE THERMAL PELVIC PHYSIOTHERAPY ACCESSORY” (100), according to claims 1 to 84, characterized by being used protected by a condom, especially during anal intercourse, in order to avoid contamination of the accessory (100) and respective complements with microorganisms and odors from the application site.

86. “PELVIC PHYSIOTHERAPY ACCESSORY WITH HEAT AND COLD OPTIONS” (100), according to claims 1 to 85, characterized by having a vibrating system (300).

87. “MULTI-PURPOSE THERMAL ACCESSORY FOR PELVIC PHYSIOTHERAPY” (100), according to claims 1 to 86, characterized in that the vibrating system (300) comprises at least one vibrating element (09), an example being, but not limited to, a transducer, or a transducer integrated into a circuit board.

88. “MULTI-PURPOSE THERMAL ACCESSORY FOR PELVIC PHYSIOTHERAPY” (100), according to claims 1 to 87, characterized in that the vibrating system (300) comprises, in addition to the vibrator (09), a control (10).

89. “MULTI-PURPOSE PELVIC PHYSIOTHERAPY THERMAL ACCESSORY” (100), according to claims 1 to 88, characterized in that the control (10) comprises at least one adjustment controller (13), examples being vibration intensity and vibration type adjustment, switching the electro-electronic circuit on and off.
